(19) United States
(12) Patent Application Publication
(10) Pub. No.: US 2026/0130584 A1
(43) Pub. Date: May 14, 2026

(54) REUSABLE LIGHT FOR MEDICAL SPECULUM

(71) Applicant: CEEK Women's Health, Inc., Portland, OR (US)

(72) Inventors: Phillip Anthony Meneau, Portland, OR (US); Chase Thompson, Portland, OR (US); Sean Mangiaracina, Portland, OR (US)

(21) Appl. No.: 19/385,826

(22) Filed: Nov. 11, 2025

Related U.S. Application Data
(60) Provisional application No. 63/720,055, filed on Nov. 13, 2024.

(51) Int. Cl. A61B 1/06; A61B 1/00; A61B 1/303; A61B 1/32; A61L 2/10

(52) U.S. Cl. CPC A61B 1/0684; A61B 1/00034; A61B 1/303; A61B 1/32; A61L 2/10; A61L 2103/15; A61L 2202/11; A61L 2202/122

(57) ABSTRACT

Disclosed are devices for use with a speculum including a reusable light and a charger for charging the reusable light. A reusable light device may include a housing, the housing having a distal end and a proximal end, wherein the proximal end of the housing defines a chamber containing: a battery, and a printed circuit board (PCB) comprising a first coil configured to charge the battery; and wherein the battery provides power to a light-emitting diode (LED) disposed at the distal end of the housing; and a clip affixed to the proximal end of the housing and configured to attach the reusable light to the medical speculum.

### RELATED APPLICATIONS

This application claims priority to, and benefit of, U.S. Provisional Ser. No. 63/720,055, filed Nov. 13, 2024, the contents of which are incorporated by reference in their entirety.

### TECHNICAL FIELD

The subject matter described herein relates generally to a reusable light for a medical speculum.

### BACKGROUND

A medical speculum is a medical tool used to provide visualization into a body cavity. Speculums or specula are traditionally used for viewing the vaginal cavity for gynecology patients. The traditional vaginal speculum consists of two bills with a hinge and a handle. The bills are inserted into the body cavity in a closed position and separated by squeezing two pieces of the handle together, thereby dilating the vagina and providing visualization and accessibility of the vagina, the cervix, and surrounding areas. Once opened, the speculum can be locked in an open position using a screw-based mechanism so an operator (e.g., physician, nurse, mid-wife, etc.) does not need to continue squeezing the pieces of the handle during the inspection. The operator can then proceed with inspecting the vagina, conducting a Pap smear or any other medical procedures that may need to be provided. Lighting devices can be affixed to specula to provide light during for the operator during the inspection or procedure.

### SUMMARY

Systems, methods, and articles of manufacture, are provided for a vaginal speculum.

According to some aspects, there is provided a reusable light for a medical speculum, the reusable light comprising: a housing, the housing having a distal end and a proximal end. The proximal end of the housing defines a chamber containing: a battery, and a printed circuit board (PCB) comprising a first coil configured to charge the battery. The battery provides power to a light-emitting diode (LED) disposed at the distal end of the housing. The reusable light further comprises a clip affixed to the proximal end of the housing and configured to attach the reusable light to the medical speculum.

In some implementations, the first coil is configured to communicate with a second coil in a charging base to inductively charge the battery. In certain implementations, the battery is configured to be charged to a desired percentage of a maximum charge of the battery. In further implementations, the housing further defines an arm, and the LED disposed at the distal end of the housing is disposed within the arm.

In some implementations, the reusable light further comprises a lens cap disposed distal the LED within the arm of the housing, the lens cap configured to prevent ingress of matter to the arm. In certain implementations, the housing comprises a polyphenylsulfone. In further implementations, the PCB comprises a button configured to turn on the reusable light upon actuation.

In some implementations, the button displays latching behavior such that repeated actuations of the button result in varying levels of brightness of the light. In certain implementations, the PCB further comprises at least a first LED configured to communicate information regarding a charge level of the battery to a user of the reusable light. In further implementations, the housing comprises a top housing half and a bottom housing half configured to couple together.

In some implementations, the battery is configured to provide power to the LED such that a brightness of the LED remains constant regardless of a charge level of the battery. In certain implementations, the lens cap comprises a flange configured to allow a snap fit between the lens cap and the arm of the reusable light. In further implementations, the PCB further comprises a microcontroller configured to customize behavior of the button.

In some implementations, the clip is affixed to the housing at a first location along a circumference of the clip. In certain implementations, the PCB comprises a memory containing customizable firmware.

In an embodiment, there is provided a kit comprising a reusable light and a base configured to charge the reusable light. In some implementations, the base further comprises an indentation having a shape substantially similar to a shape of the housing of the reusable light. In certain implementations, the reusable light is configured to charge inductively when placed in the indentation of the base. In further implementations, the base comprises a base top half and a base bottom half, the base bottom half further comprising a plate disposed therein.

In some implementations, the base comprises a USB-C port. In certain implementations, the kit further comprises a vaginal speculum. In further implementations, the kit further comprises a cord for charging the base. In certain implementations, the kit further comprises a plurality of reuseable lights.

In an embodiment, there is provided a method of using a reusable light in a gynecological procedure, the method comprising: attaching the reusable light to a vaginal speculum by the clip; actuating the button of the reusable light so as to provide light for the gynecological procedure; performing the gynecological procedure; and removing the light from the speculum.

In an embodiment, there is provided a method of charging a reusable light for a medical speculum, the method comprising: placing the reusable light for the medical speculum on a charging base, wherein the charging base comprises an indentation having a shape substantially similar to a shape of a perimeter of a housing of the reusable light, and wherein: the reusable light comprises a first coil, the charging base comprises a second coil, and the first coil is configured to inductively charge a battery of the reusable light by receiving energy from the second coil. In some implementations, the method further comprises placing the reusable light on the charging base so as to position the first coil with respect to the second coil to maximize energy transfer between the first coil and the second coil.

In an embodiment, there is provided a charger for a reusable medical light, the charger including a chamber having one or more UV-C lights configured to emit UV-C radiation and sanitize contents of the chamber, and a charging base configured to charge the reusable medical light.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations.

FIG. 1 is a front perspective view of the top of a reusable light for a medical speculum;

FIG. 2 is a back perspective view of the top of a reusable light for a medical speculum;

FIG. 3 is a perspective view of the bottom of a reusable light for a medical speculum comprising;

FIG. 4 is a front view of the reusable light for a medical speculum;

FIG. 5 is a back view of the reusable light for a medical speculum;

FIGS. 6 and 7 are side views of the reusable light for a medical speculum;

FIG. 8 is a top view of the reusable light for a medical speculum;

FIG. 9 is a bottom view of the reusable light for a medical speculum;

FIG. 10 is a perspective view of a bottom housing half of the reusable light for a medical speculum;

FIG. 11 is a perspective view of a top housing half of the reusable light for a medical speculum;

FIG. 12 is an exploded view of the lens cap, light source, battery, and printed circuit board disposed between the top housing half and the bottom housing half;

FIG. 13 is a side cross-section view of the lens cap, light source, battery, and printed circuit board disposed between the top housing half and the bottom housing half;

FIG. 14 is a front perspective view of a lens cap for use with the reusable light for a medical speculum;

FIG. 15 is a perspective view of a light pipe for use with the reusable light for a medical speculum;

FIG. 16 is a perspective view of the reusable light for a medical speculum of FIGS. 1-13 disposed in a charging base;

FIG. 17 is a perspective view of a base top half for a charging base for a reusable light for a medical speculum;

FIG. 18 is a perspective view of a base bottom half of a charging base for a reusable light for a medical speculum;

FIG. 19 is an exploded view of the base bottom half and the base top half of a charging base for a reusable light for a medical speculum;

FIG. 20 is a side cross-section view of a charging base for a reusable light for a medical speculum;

FIG. 21 is an exploded view of a printed circuit board stack for use in a reusable light for a medical speculum;

FIG. 22 is an isometric view of a printed circuit board stack for use in a reusable light for a medical speculum;

FIG. 23 is a reusable light in accordance with embodiments herein affixed to a medical speculum;

FIGS. 24A-24C are various views of a charger built in accordance with some embodiments of the present disclosure; and

FIGS. 25A-25C provide various exploded views of the charger illustrated in FIGS. 24A-24C.

When practical, similar reference numbers denote similar structures, features, or elements.

### DETAILED DESCRIPTION

Generally, a medical speculum is a medical tool used to provide visualization into a body cavity, such as the vaginal cavity, of a patient. Medical speculums may be configured to have a light source affied thereto to provide a physician and/or operator of the speculum with light during a procedure involving the speculum. Such light sources may generally be configured for single use, as the sanitation of such light sources that would be required for re-use would occupy too great an amount of a practitioner's time. Ingress of bacteria or other matter may occur during use of such a light source, which may be difficult to sanitize. Light sources can also be bent or otherwise deformed during use.

The reusable light for a medical speculum consistent with implementations of the current subject matter includes features designed for easy sanitation and charging, and designed to prevent bacteria ingress. Thus, the reusable lights described herein may provide time and cost savings to practitioners while improving patient safety due to ease of sanitation.

Additionally, a resuable light may provide lower operating costs, reduced maintenance, and a reduction in waste. Reusable lights may provide practitioners with the ability to cast light in areas of interest, which allows visualization of tissues and organs with greater clarity and detail.

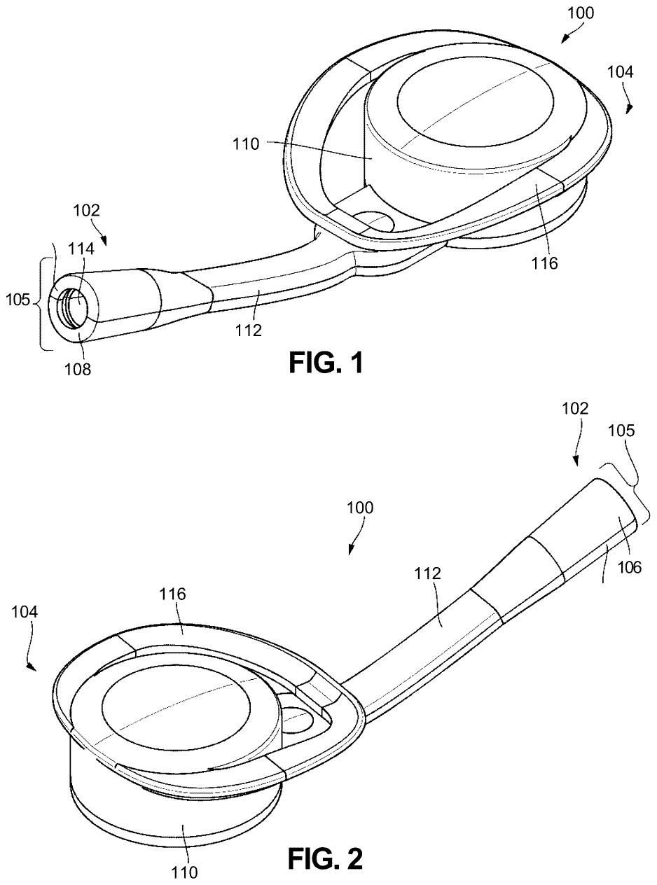

FIGS. 1-20 depict an example of a reusable light 100 and its components for use with a medical speculum (also referred to herein as the “light 100”) consistent with implementations of the current subject matter.

FIGS. 1-9 illustrate various views of a reusable light 100 for use with a medical speculum. FIG. 1 illustrates a top perspective view of reusable light 100 for use with a medical speculum, the reusable light including a distal end 102, a proximal end 104, a housing 105 comprising a top housing half 106 and a bottom housing half 108, a chamber 110, an arm 112, a light source 114, and a clip 116. The top perspective view of FIG. 1 is shown from the distal end 102 of reusable light 100. The terms “proximal” and “distal” may be used to describe herein the respective ends/sides of any of the reusable light 100 or any of its components.

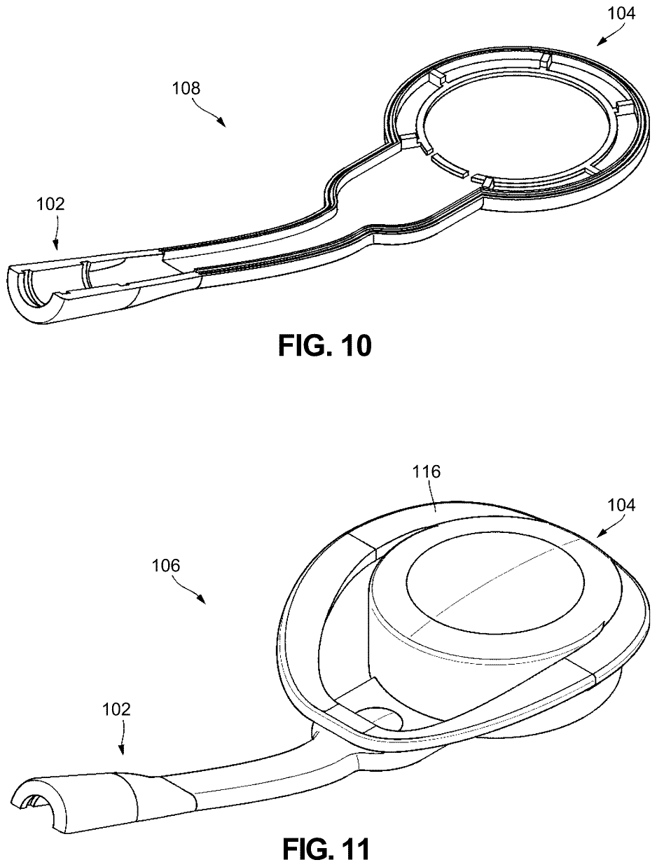

As described further with respect to FIGS. 10-13, the top housing half 106 is configured to couple to the bottom housing half 108 to define the housing 105, the chamber 110, and the arm 112. The light source 114 is disposed within the arm 112 at the distal end 102 of the reusable light 100. The light source 114 may comprise, for example, a light-emitting diode (LED). The housing comprises materials that can withstand chemicals that it may encounter during use and repeated clipping of a light thereto. For example, the housing 105 may comprise a polyphenylsulfone (PPSU) or any other similar polymer.

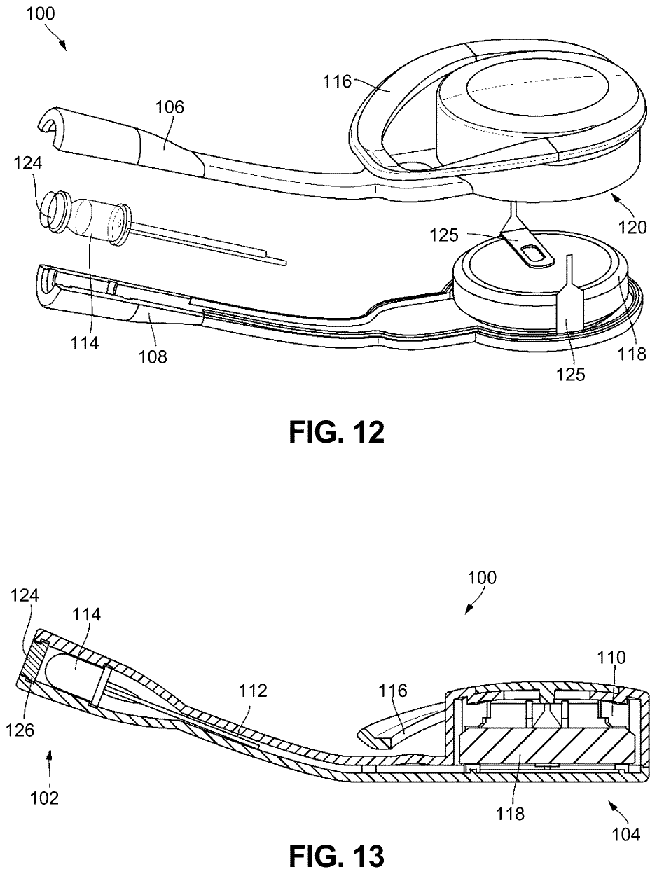

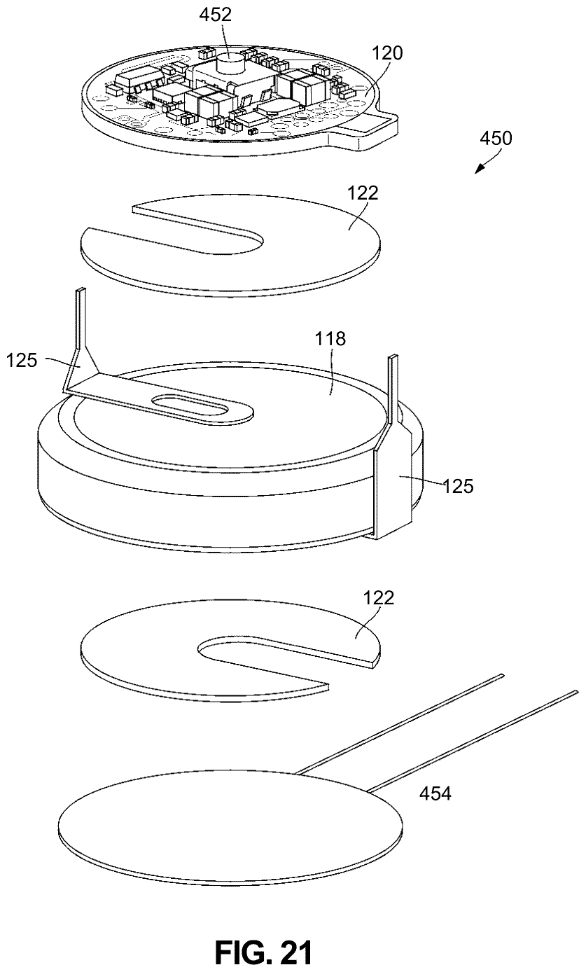

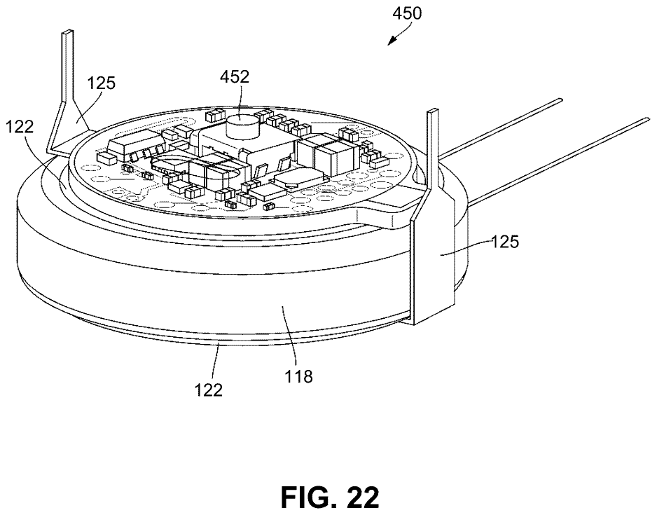

As described further with respect to FIG. 12, a battery 118 and printed circuit board (PCB) 120 (illustrated further in FIGS. 21 and 22) may be disposed in the chamber 110 defined by the top housing half 106 and the bottom housing half 108. The battery disposed within chamber 110 provides power to the light source 114 via connections disposed within the arm 112. The PCB 120 disposed within the chamber 110 provides circuitry to control the battery and the operation of the reusable light 100.

The clip 116 may have a circumference and may be generally circular, elliptical, or ovoid. The clip 116 may be affixed at a first point 119 along its circumference to the housing, for example, the top housing half 106. The clip 116 may be configured to attach to a medical speculum at a second point 121 along its circumference. The second point 121 may be free from the housing so as to provide a point of fixation or attachment to a medical speculum. The reusable light 100 may be configured to sit along a bill of a speculum so that it provides light for a practitioner without obstructing the field of view of the practitioner. For example, the arm 112 of the reusable light 100 may be configured to align along an inner surface of a top bill of a speculum so that the reusable light 100 provides light for a practitioner but remains in place, against the top bill of the speculum, during use so that the field of view of the practitioner is not obstructed.

The clip 116 has a size and comprises a material sufficient to withstand any deformation that may occur during use thereof. Such size and material thus allow the reusable light 100 to be configured for reuse. Further, the size and the material of the clip 116 are configured to keep the reusable light 100 securely affixed to a speculum during use.

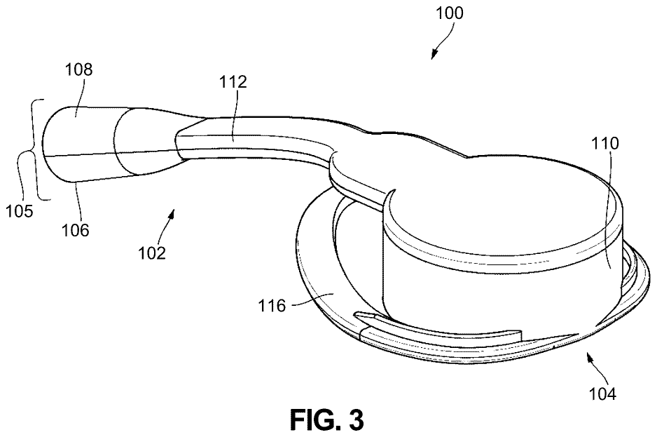

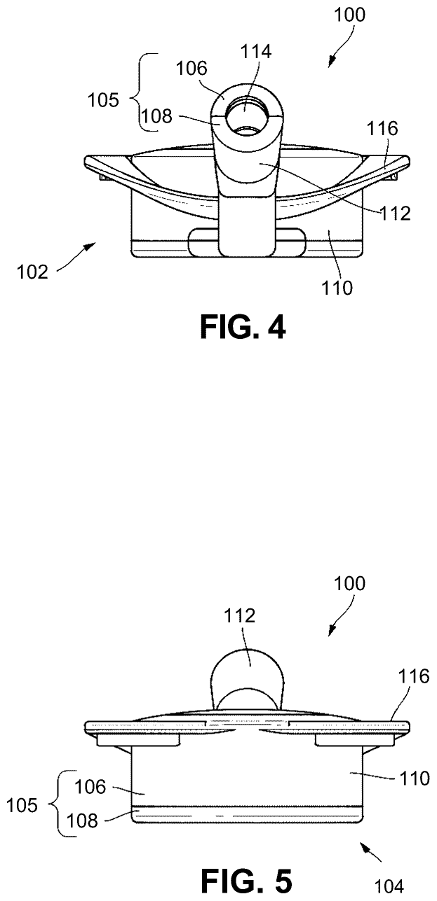

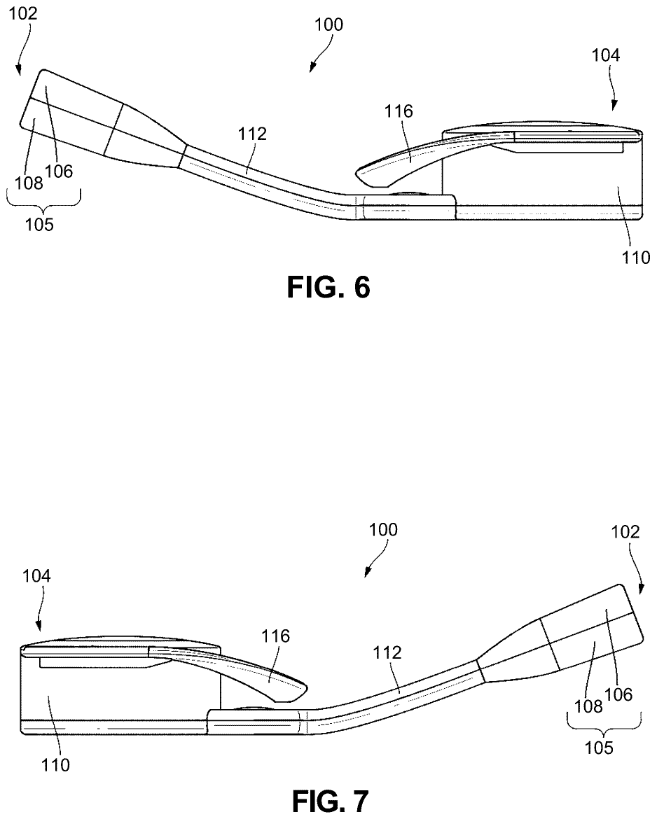

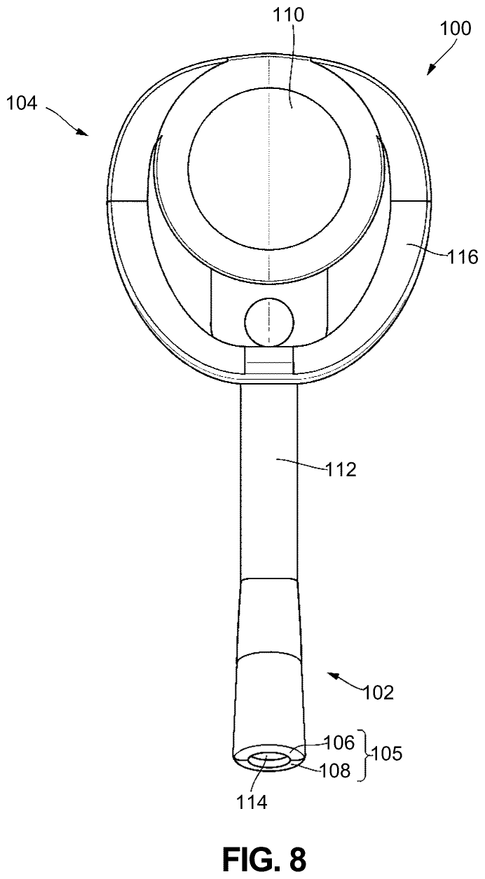

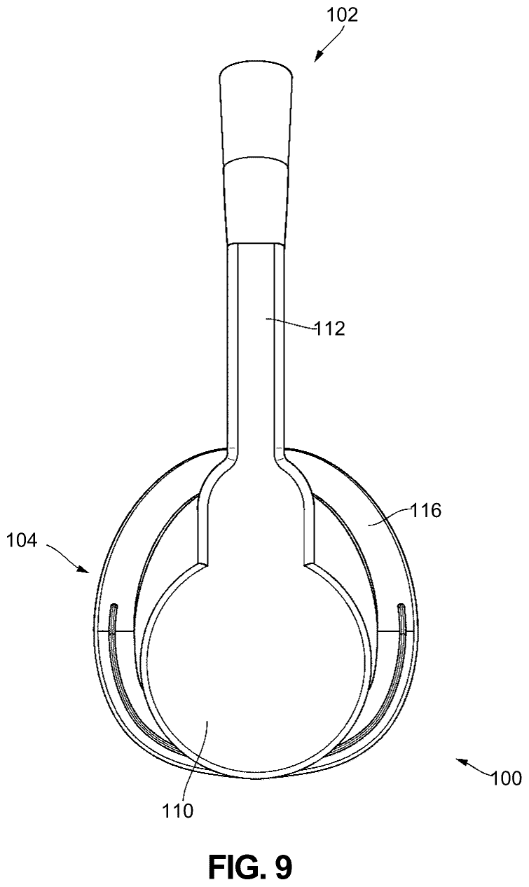

FIG. 2 shows an additional top perspective view of the reusable light 100 for a medical speculum. The top perspective view of FIG. 2 is shown from the proximal end 104 of the reusable light 100 for a medical speculum. FIG. 3 shows a bottom perspective view of the reusable light 100 for a medical speculum. The bottom perspective view of FIG. 3 is shown from the proximal end 104 of the reusable light 100 for a medical speculum. FIG. 4 shows a front view of the reusable light 100 for a medical speculum. The front view of FIG. 4 is shown from the distal end 102 of reusable light 100. FIG. 5 shows a back view of the reusable light 100 for a medical speculum. FIGS. 6 and 7 show side views of the reusable light 100 for a medical speculum. FIG. 8 shows a top view of the reusable light 100 for a medical speculum. FIG. 9 shows a bottom view of the reusable light 100 for a medical speculum.

FIG. 10 shows a front perspective view of the bottom housing half 108 of the reusable light 100 for a medical speculum. FIG. 11 shows a front perspective view of the top housing half 106 of the reusable light 100 for a medical speculum.

FIG. 12 shows an illustrative embodiment of how the top housing half 106 and the bottom housing half 108 of the reusable light for a medical speculum may be configured to couple so as to define the housing 105, the arm 112, and the chamber 110. In some implementations, the top housing half 106 and the bottom housing half 108 are welded together ultrasonically to form housing 105. The light source 114 may be configured so as to be disposed within the arm 112 defined by the coupling of the top housing half 106 and the bottom housing half 108. A lens cap 124 may be disposed at the distal most end of the arm 112 so as to protect the light source 114 and the arm 112 from bacteria ingress during use of the reusable light.

The battery 118 and PCB 120 may be configured so as to be disposed within the chamber 110 defined by the coupling of the top housing half 106 and the bottom housing half 108. The battery 118 and the PCB 120 may provide the necessary components and circuitry so as to power the light source 114 and turn the reusable light 100 on and off. As shown, the battery 118 has a flat top side 123 configured to abut the PCB 120. As described below with respect to FIG. 21, the PCB 120 may comprise contact pads 122 to provide contact between the PCB 120 and the battery 118. The contact pads 122 may be soldered to the PCB 120.

In some implementations, the PCB contains a button configured to be actuated upon pressing the top of the proximal end 104 of the housing 105. Upon actuation, the button may turn the reusable light 100 on or off. The button may display latching behavior such that repeated presses of the button result in varying levels of brightness of the reusable light. For example, a first press of the button may turn on the reusable light 100, a second press may make the reusable light brighter, and a third press may turn the reusable light off. A microcontroller of the PCB 120 can customize button behavior. In general, a button with customizable latching behavior can be configured so as to allow any number of varying levels of brightness to be implemented by the reusable light 100.

The PCB 120 may comprise a memory containing customizable firmware. For example, the firmware may be customizable according to a practitioner's needs. The firmware may be customizable to allow for the reusable light 100 to auto-turn off after a particular period of time. Such customizable features of the firmware of the PCB 120 help to promote longevity of the reusable light 100. The PCB 120 may further comprise circuitry to enable short-range communication or Bluetooth capability of the reusable light 100.

The PCB 120 may, in some implementations, contain a light or a plurality of lights that communicate information about the status of the reusable light 100 to a user thereof. Such information may be communicated via light pipes that provide an optical passageway from the PCB to the user. For example, different light-emitting diodes (LEDs) on the PCB 120 may comprise different colors that may represent battery status. In some implementations, a red light may represent a near-empty battery, a yellow light may represent a half-full battery, and a green light may represent a battery charged to any desired percentage of the maximum. Any number of the LEDs disposed on the PCB may be configured to blink when the battery 118 is running low. Any number of filters can be applied to change the color temperature of the light communicated by the light pipe.

The battery 118 may be configured to couple via clips 125 to the chamber 110 of the housing 105.

In some implementations, the battery 118 and PCB 120 are configured to provide power to the light source 114 such that the light provided by the light source 114 remains constant in its intensity and/or brightness regardless of the amount of energy stored in the battery 118. Ensuring that the light provided by the light source 114 remains constant in intensity and/or brightness regardless of the battery level helps to ensure a smooth experience for the operator of the reusable light.

FIG. 13 shows a cross-section of the reusable light 100 for a medical speculum showing the battery 118 disposed in the chamber 110 and the light source 114 disposed in the arm 112, both the chamber 110 and the arm 112 being defined by the coupling of the top housing half 106 to the bottom housing half 108. The lens cap 124 disposed at the distal end 102 of the reusable light 100 protects the light source 114 from bacteria ingress.

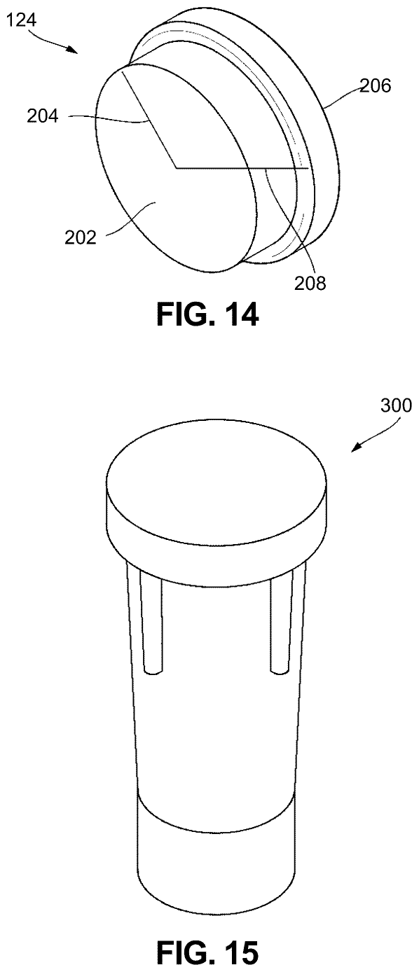

FIG. 14 shows a front perspective view of the lens cap 124. The lens cap 124 may have a first portion 202 having a first radius 204 and a second portion 206 having a second radius 208. The first radius 204 and the second radius 208 may define a flange that is configured to fit into a groove 126 (as shown in FIG. 13) in the arm 112 of the housing 105 such that a snap fit between the lens cap 124 and the distal end 102 of the reusable light 100 prevents ingress of bacteria into the arm 112 of the reusable light.

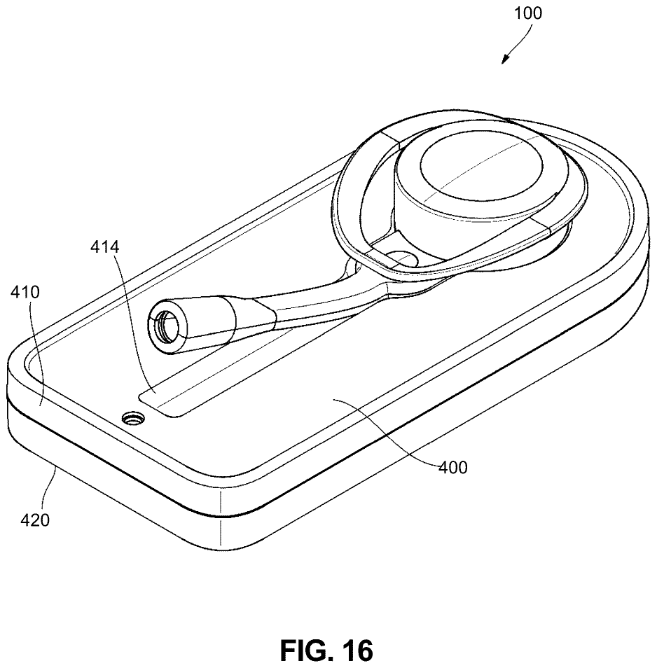

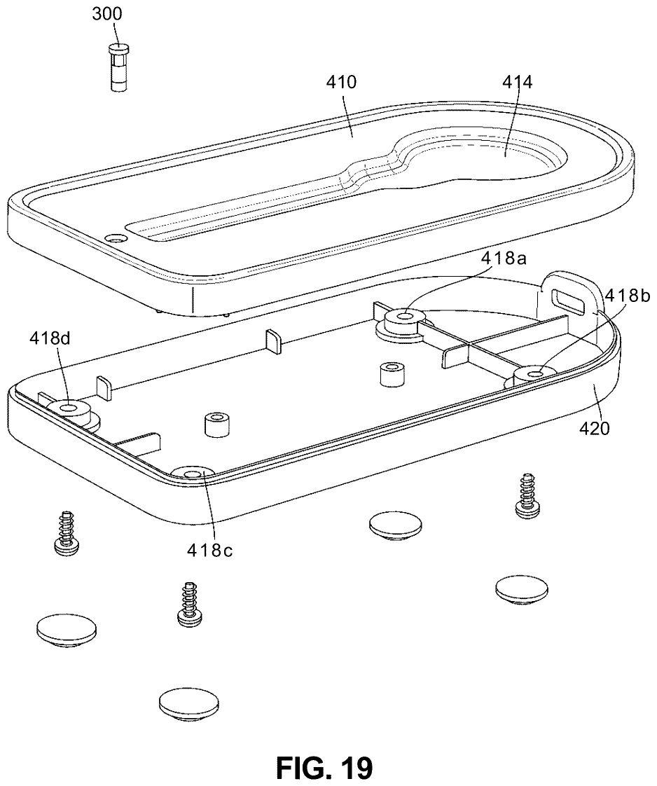

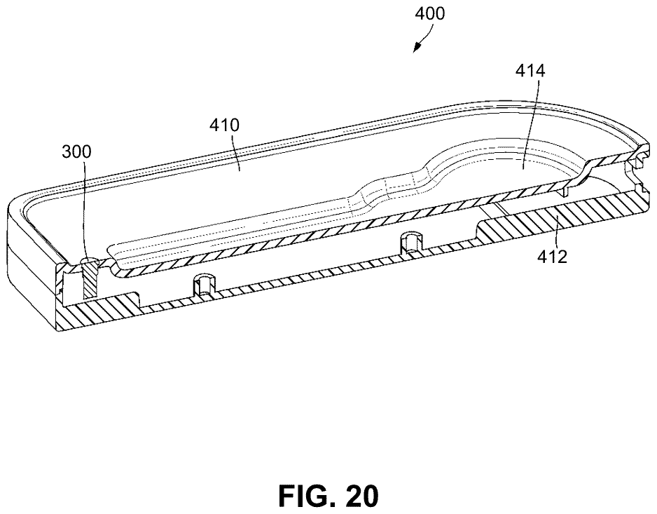

FIG. 15 shows a light pipe 300 that can be used to communicate information from the LEDs of PCB 120 to a user of the reusable light 100. The light pipe can be press-fit into the top of the housing 105 of the reusable light 100. As described further below with respect to FIGS. 19 and 20, a light pipe, such as light pipe 300, can also be placed in the charging base 400 of FIGS. 16-20.

FIG. 16 shows the reusable light 100 for a medical speculum of FIGS. 1-13 disposed in a charging base 400. Charging base 400 may be configured with the necessary circuitry, including an inductive coil, for wirelessly providing power to the reusable light 100 when the reusable light 100 is disposed on the charging base 400. Charging base 400 may be configured to receive power via a USB-C port (not illustrated), or by any other charging means that allow charging base 400 to remain portable. The reusable light 100 and the base 400 may comprise or be sold together as a kit.

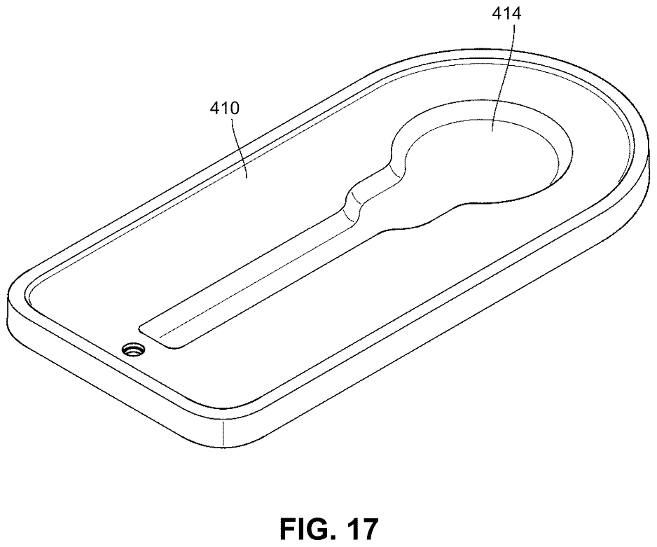

As described further with respect to FIGS. 17-20, charging base 400 comprises a base top half 410 and a base bottom half 420. Base top half 410 may be configured with an indentation 414 having a substantially similar shape along its perimeter to that of bottom housing half 108 of the reusable light 100. As used herein, a “substantially similar shape” may mean a shape that is “similar” in the geometric sense (i.e., two shapes differ only in scale such that their sizes may be different while the angles defined between their own sides remains the same). By ensuring similarity in shape between the bottom housing half 108 of the reusable light 100 and the indentation 414, positioning of the reusable light 100 once disposed on the charging base 400 can be facilitated such that the maximum amount of power is transferred between the inductive charging coils of the base 400 and the reusable light 100. Further, the base 400 may comprise a plate made of a material with substantial mass (e.g., steel) such that disturbances to the base 400 during charging of the reusable light 100 do not disrupt the positioning of the reusable light 100.

In some implementations, the inductive coils in the base 400 and in the reusable light 100 are configured to transfer energy to the battery 118 such that the battery 118 is only charged to a desired percentage of its maximum charge. For example, the inductive coils in the base 400 and in the reusable light 100 may cooperate so as to charge the battery 118 to 85 percent, 90 percent, or 95 percent of the maximum charge. The longevity of the battery 118 can be extended by charging the battery only to a desired percentage of the maximum charge. Battery 118 may also be configured with a customizable or variable voltage to allow an end user of the reusable light 100 to ensure that sufficient light can be provided by reusable light 100 for their intended procedures.

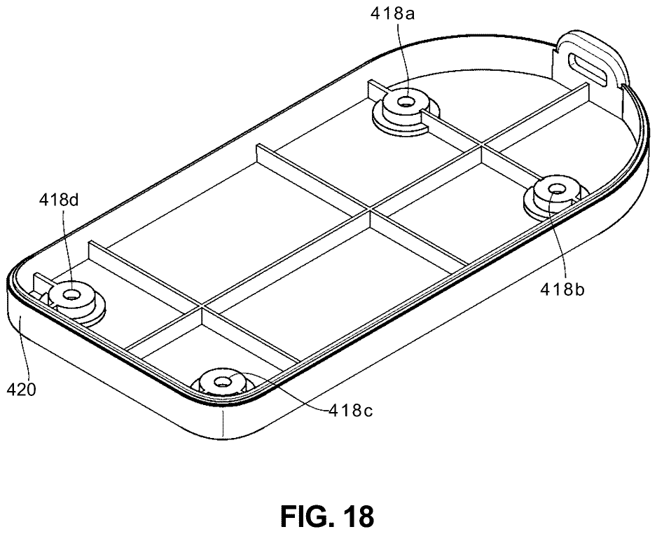

FIG. 17 shows the base top half 410 having indentation 414. FIG. 18 shows the base bottom half 412 configured to couple to the base top half 410. Base bottom half 412 comprises a plurality of screw holes 418a, 418b, 418c, 418d. FIG. 19 illustrates how the plurality of screw holes 418a, 418b, 418c, 418d are configured to receive screws to coupled the base bottom half 412 to the base top half 410. FIG. 19 also illustrates a light pipe 300 that can be used to communicate information about the charging of the reusable light 100 to a user when the reusable light 100 is placed in the indentation 414. FIG. 20 shows a cross-section view of the charging base 400 comprising base top half 410 coupled to base bottom half 412. Light pipe 300 of FIG. 20 can be used communicate information regarding the charging status of the reusable light 100 to a user when the reusable light 100 is placed in the indentation 414 of the charging base 400.

FIG. 21 illustrates an exploded view of a printed circuit board stack 450 in accordance with embodiments of the reusable light 100 described herein. The printed circuit board stack 450 of FIG. 21 comprises a battery 118 configured to power the reusable light 100 and a printed circuit board (PCB) 120 with the functionalities described above with respect to FIG. 12. The printed circuit board stack 450 of FIG. 21 also comprises spacers 122 and clips 125. The spacers 122 may comprise foam spacers.

In particular, PCB 120 comprises a button 452 configured to turn the reusable light 100 off and on. As described, the button 452 may display latching behavior such that repeated presses of the button 452 result in varying levels of brightness of the reusable light 100. For example, a first press of the button 452 may turn on the reusable light 100, a second press of the button 452 may make the reusable light brighter, and a third press of the button 452 may turn the reusable light 100 off. A microcontroller of the PCB 120 can customize button behavior. In general, a button 452 with customizable latching behavior can be configured so as to allow any number of varying levels of brightness to be implemented by the reusable light 100.

The PCB stack 450 further comprises a first inductive coil 454. The first inductive coil 454 is configured to receive electrical energy from a second conductive coil disposed within a charging base, such as the charging base 400 of FIGS. 16-20.

The PCB 120 may comprise a memory containing customizable firmware. For example, the firmware may be customizable according to a practitioner's needs. The firmware may be customizable to allow for the reusable light 100 to auto-turn off after a particular period of time. Such customizable features of the firmware of the PCB 120 help to promote longevity of the reusable light 100. The PCB 120 may further comprise circuitry to enable short-range communication or Bluetooth capability of the reusable light 100.

The PCB 120 may, in some implementations, contain a light or a plurality of lights that communicate information about the status of the reusable light 100 to a user thereof. Such information may be communicated via light pipes that provide an optical passageway from the PCB to the user. For example, different light-emitting diodes (LEDs) on the PCB 120 may comprise different colors that may represent battery status. In some implementations, a red light may represent a near-empty battery, a yellow light may represent a half-full battery, and a green light may represent a battery charged to any desired percentage of the maximum. Any number of the LEDs disposed on the PCB may be configured to blink when the battery 118 is running low. Any number of filters can be applied to change the color temperature of the light communicated by the light pipe. FIG. 22 shows an isometric view of PCB stack 450, comprising the PCB 120, the battery 118, the clips 125 and the contact pads 122.

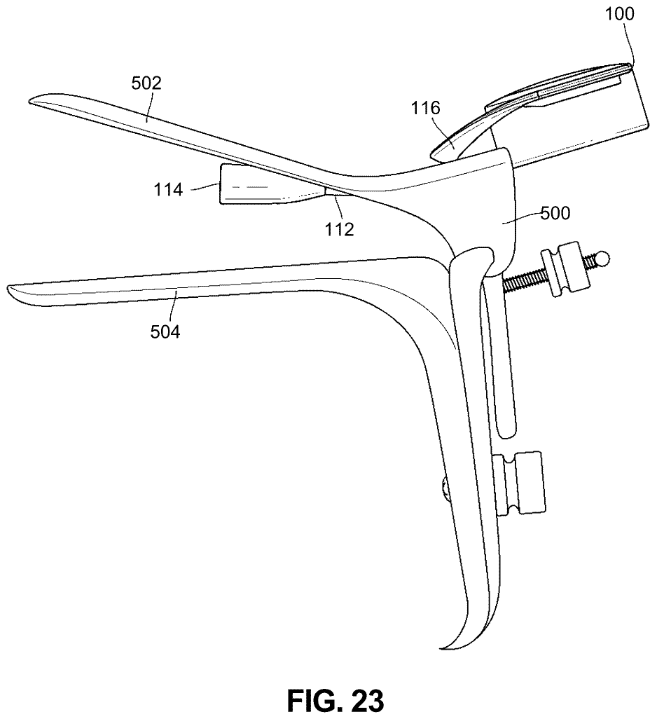

FIG. 23 shows the reusable light 100 affixed to a medical speculum 500. The medical speculum 500 of FIG. 23 comprises a first bill 502 and a second bill 504, though the reusable light 100 described herein is configured to attach to a medical speculum with any appropriate number of bills. As discussed, the reusable light 100 may be configured to sit along a bill of the speculum 500, such as first bill 502, so that it provides light from light source 114 for a practitioner without obstructing the field of view of the practitioner. The reusable light 100 has a clip 116 configured to attach the reusable light 100 to the speculum 500 such that the arm 112 of the reusable light 100 extends along a surface of the first bill 502. By placing the arm 112 along a surface of the first bill, the light source 114 of the reusable light 100 can provide illumination for an operator of the speculum 500 without comprising the field of view of the operator.

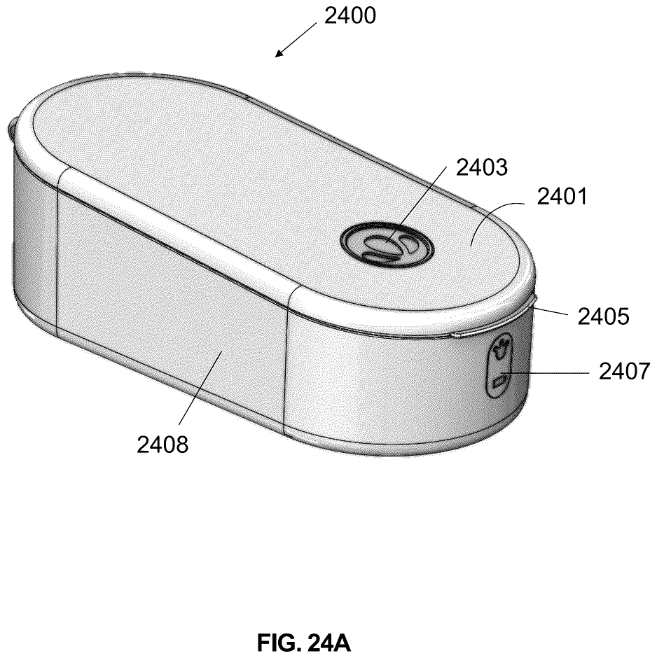

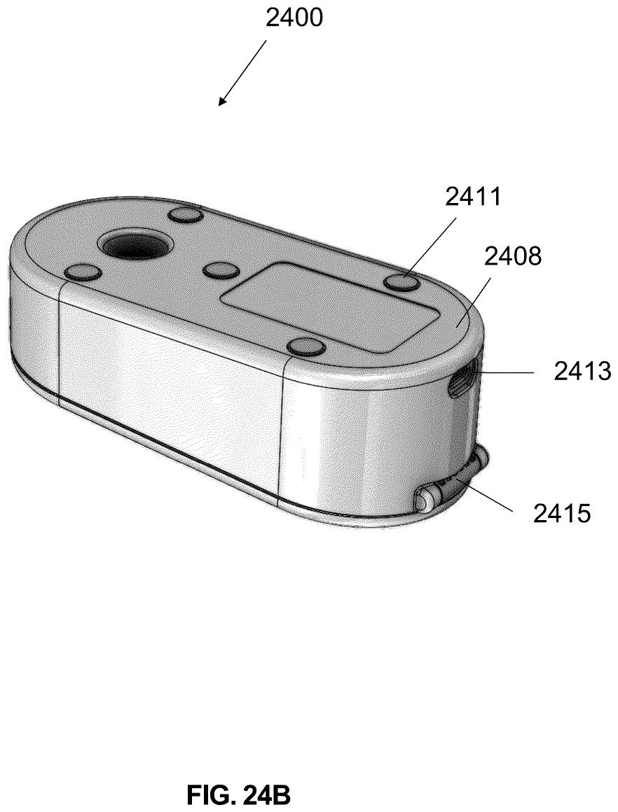

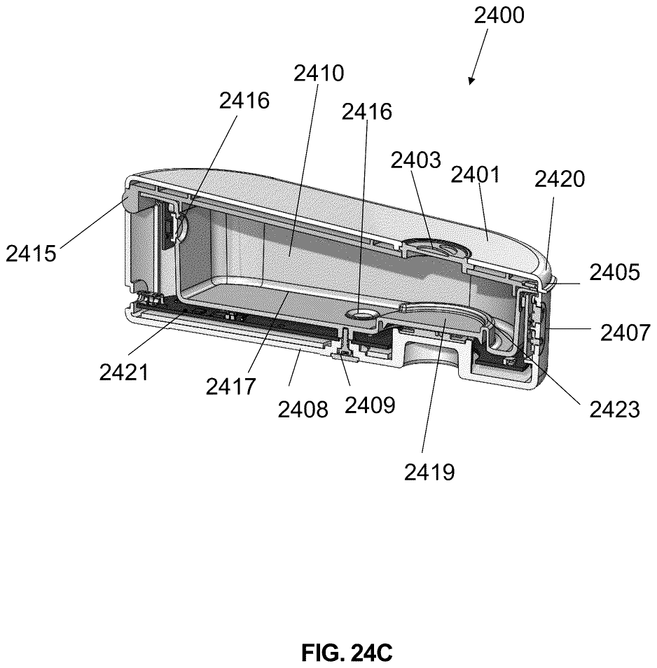

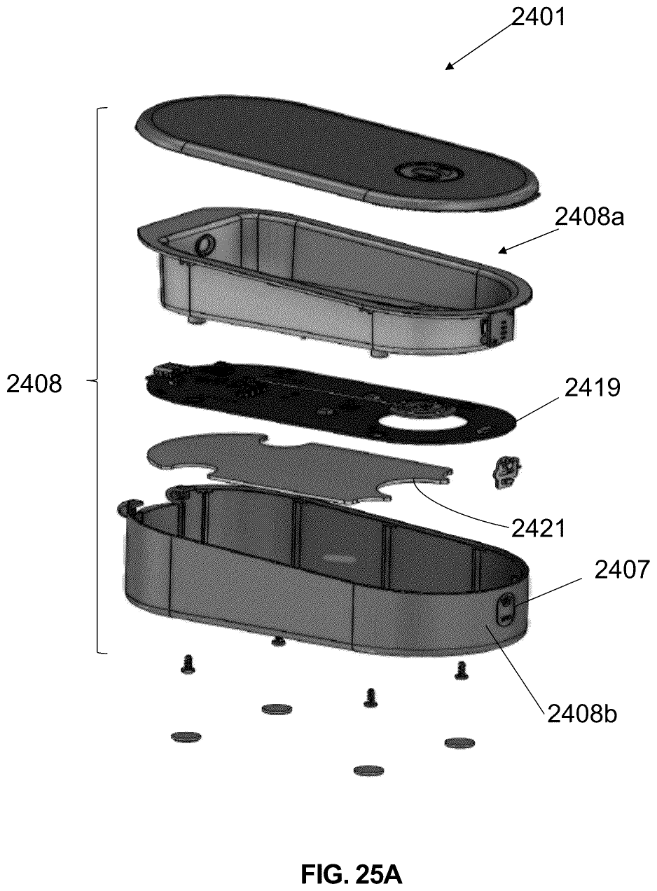

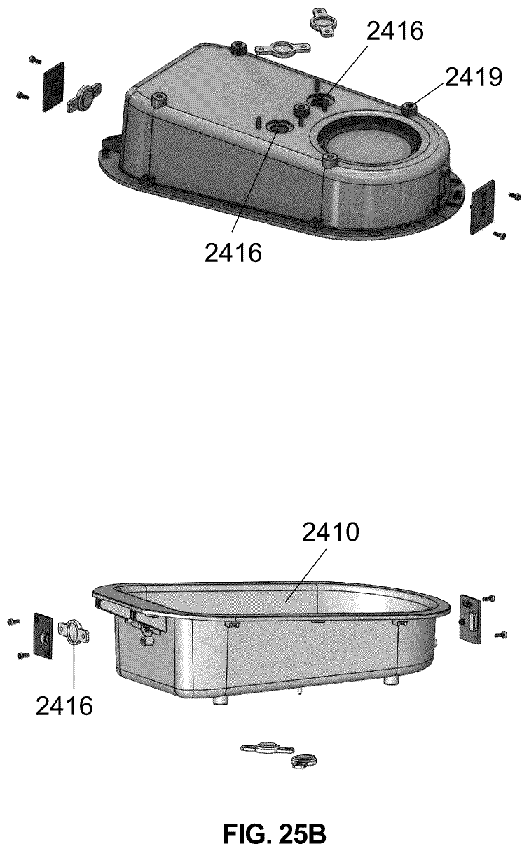

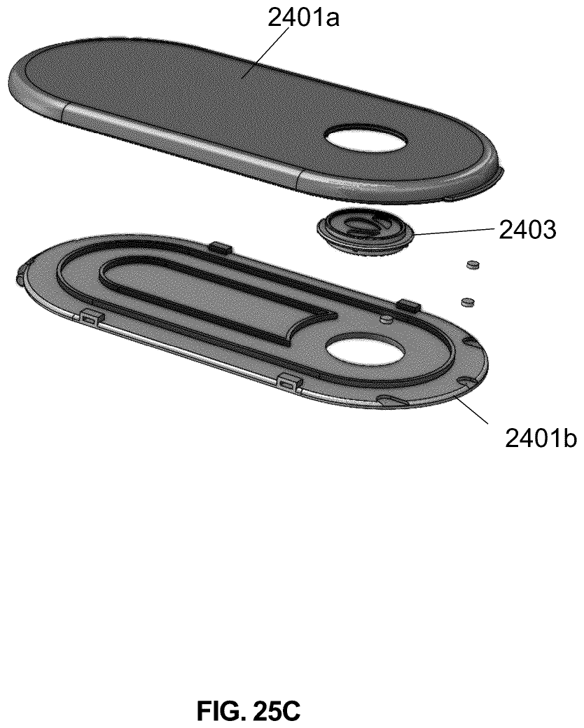

FIGS. 24A-25C provide illustrations of an additional embodiment of a charger that can be used in connection with a reusable light such as reusable light 100.

FIG. 24A provides a first perspective view of the charger 2400. FIG. 24B provides a second perspective, rear view of the charger 2400. FIG. 24C provides a cross-sectional view of the charger 2400.

As illustrated in FIGS. 24A-24C in some embodiments, the charger 2400 can form an enclosed area or chamber 2410 that is configured to receive a reusable light. For example the charger 2400 may be in the shape of a rectangular prism with a slanted top and rounded edges. Alternative shapes are envisioned.

In some implementations, the charger 2400 may be sized to hold a reusable light such as reusable light 100 within a chamber in the interior of the charger 2400. The charger 2400 may include a top lid surface 2401 and a bottom frame 2408. In some implementations, the top lid surface 2401 and the bottom frame 2408 may be connected via hinge 2415. In some implementations, the top lid surface 2401 can be rotated upward about the hinge 2415 to open the charger 2400 such as when placing or removing a reusable light into or from the chamber 2410.

In some implementations, the top lid 2401 and the bottom frame 2408 can be attached via a magnet 2420 that is configured to be trapped between the two halves of the lid. In some embodiments a tab 2405 can be provided for a user to lift the top lid 2401 of the charger 2400 with respect to the bottom frame 2408.

In some implementations, the interior chamber 2410 of the charger 2400 may include one or more additional lights such as UV-C lights 2416 that are configured to disinfect and/or sterilize the reusable light. For example, as shown in FIG. 24C, a first UV-C light 2416 can be positioned proximate the hinge on a wall of the charger container. The first UV-C light 2416 can be configured to shine UV-C disinfecting light to the interior of the chamber 2410 where a reusable light can be positioned. In the illustrated embodiment, two additional UV-C lights 2416 are positioned in proximity to a charger base 2419.

In some implementations, the charger base 2419 may include a collar 2423 that is configured to orient and position the reusable light such that UV-C light is applied to the appropriate areas of the reusable light.

In some implementations, a vertically mounted PCB with a hall effect sensor can be positioned adjacent and below the magnet 2420 configured to be trapped between the two halves of the lid. Accordingly, when the lid is closed, the magnet and hall effect sensor can produce a signal for the PCB to start charging the reusable light and/or enable the UV-C lights to disinfect the reusable light. In some implementations, a microcontroller is communicatively coupled to the PCB and/or hall effect sensor to control operation of the reusable light and/or UV-C lights.

In addition to the magnet 2420 positioned at the intersection of the lids that enables charging and disinfection of the reusable light via a hall effect sensor, additional magnets can be positioned across the top lid surface 2401 with corresponding magnets in the bottom frame 2403 in order to securely close the lid of the top lid surface 2401 with respect to the bottom frame 2408.

In some implementations, the microcontroller may be coupled to indicators 2407 which are configured to display a light. The indicators 2407 may provide an indication that the charger is on or off, or that the reusable light is fully charged or partially charged, and the like. For example, the indicators 2407 may blink or be steady, emit varying pulses, or have different colors or the like to convey information regarding the charging status of the reusable light and/or the disinfectant status of the chamber and/or reusable light. In some implementations, the microcontroller may be loaded with software configured to program the timing period that the UV-C lights apply UV-C. For example, the microcontroller may be programmed to emit UV-C light for 1 minute, 5 minutes, 10 minutes, 20 minutes, 1 hour, etc. or any other suitable time period for disinfecting the reusable light. In some embodiments, as shown in FIG. 24, at least one exterior wall of the charger 2400 may include indicator 2407 that are configured to show whether cleaning is completed, in progress, if the device is fully charged, partially charged, or the like.

In some implementations, the microcontroller can be configured to detect when power usage is below a threshold to recognize that charging has stopped. Accordingly, the microcontroller may control operation of the indicator 2407 and change the lighting responsive to recognizing that the power usage has dropped below a threshold value.

In some implementations, a window 2403 may be translucent and allow a user to visualize whether a UV-C light is being applied to the reusable light. In this manner, a user may have visibility into the chamber of the charger to confirm proper usage of the device without exposing the user to UV-C radiation, which may be carcinogenic. The window 2403 may be composed of a lens or plastic. The window plastic 2403 may be configured to include a plastic that does not allow UV-C to pass through.

The charger 2400 may include a charging port 2413 positioned along an exterior surface of the charger 2400. In some implementations, the charging port can be configured to connect to a USB power cord and the like.

In some implementations, a bottom surface of the bottom frame 2408 may include one or more feet or pads 2411 to prevent friction or movement across a surface the charger 2400 is placed on.

FIGS. 25A-25C provide exploded views of the charger illustrated in FIGS. 24A-24C. For example, FIG. 25A provides an exploded view of the fully assembly including the lid 2401 and bottom frame 2408. FIG. 25B provides an exploded view of the internal subassembly for the bottom frame 2408. FIG. 25C provides an exploded view of the lid 2401 subassembly.

In some implementations, the lid 2401 and bottom frame 2408 are composed of plastic. Lids and other components forming the frame of the charger 2400 can attach to other components such as the windows, LED lights, UV-Cs, plates, bases, subassembly components using pressure fits, screws, and other means.

In the illustrated embodiment, the bottom frame 2408 can include an internal subassembly 2408a which is attached to a second plastic piece 2408c with a charging base 2419 and a weighted metal piece 2421 placed therebetween. The weighted metal piece 2421 can be composed of steel or other suitable materials that can be configured to keep the charger in the proper position when the top lid is opened, preventing the charger from tipping over or flipping.

In the illustrated embodiment, the lid 2401 can be composed of two plastic subunits 2401a and 2401b which may be pressure fit, snap fit, or screwed together. The two plastic subunits may engage to securely hold a lens to form window 2403.

The interior components and/or walls of the charger 2400 forming the chamber 2410 may be painted a particular color in order to increase its effectiveness of cleaning using UV-C radiation. For example, the color of the walls needs to be white or other suitable colors to allow UV-C radiation to reflect and bounce within the chamber, without being absorbed by the frame of the charger.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. For example, the logic flows may include different and/or additional operations than shown without departing from the scope of the present disclosure. One or more operations of the logic flows may be repeated and/or omitted without departing from the scope of the present disclosure. Other implementations may be within the scope of the following claims.

We claim:

1. A reusable light for a medical speculum, the reusable light comprising:
a housing, the housing having a distal end and a proximal end, wherein the proximal end of the housing defines a chamber containing:
a battery, and
a printed circuit board (PCB) comprising a first coil configured to charge the battery; and wherein
the battery provides power to a light-emitting diode (LED) disposed at the distal end of the housing; and
a clip affixed to the proximal end of the housing and configured to attach the reusable light to the medical speculum.

2. The reusable light of claim 1, wherein the first coil is configured to communicate with a second coil in a charging base to inductively charge the battery.

3. The reusable light of claim 1 or 2, wherein the battery is configured to be charged to a desired percentage of a maximum charge of the battery.

4. The reusable light of any one of claims 1-3, wherein
the housing further defines an arm, and
the LED disposed at the distal end of the housing is disposed within the arm.

5. The reusable light of any one of claims 1-4, further comprising a lens cap disposed distal the LED within the arm of the housing, the lens cap configured to prevent ingress of matter to the arm.

6. The reusable light of any one of claims 1-5, wherein the housing comprises a polyphenylsulfone.

7. The reusable light of any one of claims 1-6, wherein the PCB comprises a button configured to turn on the reusable light upon actuation.

8. The reusable light of any one of claims 1-7, wherein the button displays latching behavior such that repeated actuations of the button result in varying levels of brightness of the light.

9. The reusable light of any one of claims 1-8, wherein the PCB further comprises at least a first LED configured to communicate information regarding a charge level of the battery to a user of the reusable light.

10. The reusable light of any one of claims 1-9, wherein the housing comprises a top housing half and a bottom housing half configured to couple together.

11. The reusable light of any one of claims 1-10, wherein the battery is configured to provide power to the LED such that a brightness of the LED remains constant regardless of a charge level of the battery.

12. The reusable light of any one of claims 1-11, wherein the lens cap comprises a flange configured to allow a snap fit between the lens cap and the arm of the reusable light.

13. The reusable light of any one of claims 1-12, wherein the PCB further comprises a microcontroller configured to customize behavior of the button.

14. The reusable light of any one of claims 1-13, wherein the clip is affixed to the housing at a first location along a circumference of the clip.

15. The reusable light of any one of claims 1-14, wherein the PCB comprises a memory containing customizable firmware.

16. A kit comprising the reusable light of any one of claims 1 to 15 and a base configured to charge the reusable light.

17. The kit of claim 16, wherein the base further comprises an indentation having a shape substantially similar to a shape of the housing of the reusable light.

18. The kit of claim 16 or 17, wherein the reusable light is configured to charge inductively when placed in the indentation of the base.

19. The kit of any one of claims 16-18, wherein the base comprises a base top half and a base bottom half, the base bottom half further comprising a plate disposed therein.

20. The kit of any one of claims 16-19, wherein the base comprises a USB-C port.

21. The kit of any one of claims 16 to 20, further comprising a vaginal speculum.

22. The kit of any one of claims 16 to 21, further comprising a cord for charging the base.

23. The kit of any one of claims 16 to 22, comprising a plurality of reuseable lights.

24. A method of using the reusable light of any of claims 1-15 in a gynecological procedure, the method comprising:
attaching the reusable light to a vaginal speculum by the clip;
actuating the button of the reusable light so as to provide light for the gynecological procedure;
performing the gynecological procedure; and
removing the light from the speculum.

25. A method of charging a reusable light for a medical speculum, the method comprising:
placing the reusable light for the medical speculum on a charging base, wherein the charging base comprises an indentation having a shape substantially similar to a shape of a perimeter of a housing of the reusable light, and wherein:
the reusable light comprises a first coil,
the charging base comprises a second coil, and
the first coil is configured to inductively charge a battery of the reusable light by receiving energy from the second coil.

26. The method of claim 25, further comprising placing the reusable light on the charging base so as to position the first coil with respect to the second coil to maximize energy transfer between the first coil and the second coil.

27. A charger for a reusable medical light, the charger comprising:
a chamber comprising one or more UV-C lights configured to emit UV-C radiation and sanitize contents of the chamber; and
a charging base configured to charge the reusable medical light.

28. The charger of claim 27 further comprising:
wherein the charging base comprises a collar configured to position and orient the reusable medical light.

29. The charger of claim 27 further comprising:
a lid; and
a bottom frame, wherein the bottom frame and the lid form the chamber.

30. The charger of claim 29, wherein the lid and the bottom frame engage via at least one magnet.

31. The charger of claim 30, wherein the magnet induces a microcontroller to change a light on an exterior wall of the chamber.

32. The charger of claim 30, wherein the magnet induces a microcontroller to control operation of the one or more UV-C lights.

33. The charger of claim 27, wherein the reusable medical light comprises an inductive charging mechanism.

34. The charger of claim 29, wherein the lid comprises a window for visualization of the chamber.

35. The charger of claim 27, wherein the chamber comprises three UV-C lights.

36. The charger of claim 35, wherein a first UV-C light is positioned on an interior wall of the chamber opposite the wall proximate the charging base.

37. The charger of claim 36, wherein at least one UV-C light is positioned on a surface proximate the charging base on a bottom interior surface of the chamber.

38. A kit comprising:
a charger of claim 27; and
a reusable light for a speculum.
